(19) United States
(12) Patent Application Publication
(10) Pub. No.: US 2026/0021064 A1
(43) Pub. Date: Jan. 22, 2026

(54) ACETAMINOPHEN AND NAPROXEN FOR TREATING PAIN

(71) Applicant: Kenvue Brands LLC, Summit, NJ (US)

(72) Inventors: Julia COSKEY, Lansdale, PA (US); Stephen A. ULRICH, Cherry Hill, NJ (US); Steven SACAVAGE, Glenside, PA (US)

(21) Appl. No.: 19/120,844

(22) PCT Filed: Oct. 31, 2023
(86) PCT No.: PCT/US2023/078269

§ 371 Date: Apr. 14, 2025

Related U.S. Application Data
(60) Provisional application No. 63/422,547, filed on Nov. 4, 2022.
(60) Provisional application No. 63/504,237, filed on May 25, 2023.

(51) Int. Cl. A61K 31/192; A61K 9/20; A61K 9/28; A61K 31/167; A61P 29/00

(52) U.S. Cl. CPC A61K 31/192; A61K 9/2018; A61K 9/2027; A61K 9/2054; A61K 9/2059; A61K 9/2077; A61K 9/2833; A61K 9/2893; A61K 31/167; A61P 29/00

(57) ABSTRACT

The disclosure is directed to improved methods of treating minor pain in humans using a pharmaceutical dosage form comprising acetaminophen in admixture with naproxen.

### CROSS-REFERENCE TO RELATED APPLICATIONS

This application claims the benefit of U.S. Provisional Application Nos. 63/422,547 filed Nov. 4, 2022, and 63/504,237 filed May 25, 2023, the entireties of which are incorporated by reference herein.

### TECHNICAL FIELD

The disclosure is directed to improved methods of treating minor pain in humans using a pharmaceutical dosage form comprising acetaminophen in admixture with naproxen.

### BACKGROUND

Acetaminophen is a commercially available analgesic. Naproxen sodium is a commercially available nonsteroidal anti-inflammatory drug (NSAID). Each has been described, individually, as a safe and effective over-the-counter (OTC) treatment for minor pain. Acetaminophen has been combined with narcotics, for example, opioids such as hydrocodone, codeine, and oxycodone, to increase pain relief, but these combination products may be contraindicated for certain patient populations. There still exists a need, however, for a safe and effective OTC treatment that can induce pain relief.

### SUMMARY

The disclosure is directed to methods of inducing minor pain relief in a human in need thereof. According to these methods, the human is administered a pharmaceutical dosage form comprising acetaminophen in admixture with naproxen sodium, wherein the weight ratio of the acetaminophen to the naproxen sodium is from about 2:1 to about 4:1. Pharmaceutical dosage forms useful in these methods are described herein, as well as methods of their preparation.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination. Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

In the disclosure, the singular forms “a,” “an,” and “the” include the plural reference, and reference to a particular numerical value includes at least that particular value, unless the context clearly indicates otherwise. Thus, e.g., a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. In some cases, the descriptor “about” will be understood as meaning ±up to 10%, for example ±10, 9, 8, 7, 6, 5, 4, 3, 2 or 1%. Where present, all ranges are inclusive and combinable. That is, references to values stated in ranges include every value within that range.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude an optional element. As such, this statement is intended to serve as antecedent basis for use of such exclusive terminology as “only” and the like in connection with the recitation of claim elements, or use of a “negative” limitation. Finally, while an embodiment may be described as part of a series of steps or part of a more general structure, each said step may also be considered an independent embodiment in itself.

The disclosure is directed to methods of inducing pain relief to humans, e.g. those at least 12 years old, at least 16 years old, or at least 18 years old, experiencing minor pain, by orally administering a pharmaceutical dosage form comprising acetaminophen (i.e., paracetamol) in admixture with naproxen, which is preferably in the form of a pharmaceutically acceptable salt of naproxen such as naproxen sodium.

“Pharmaceutical dosage form” refers to any orally-ingestible form, for example, solid, semi-solid, or liquid. In some aspects, the dosage forms are solid, though they may include liquid or semi-solid ingredients. Preferred dosage forms are tablets (e.g., compressed or molded solid dosage forms of any shape or size), preferably film-coated tablets. Other dosage forms include capsules and powders.

As used herein, “minor pain” includes, e.g., headache, backache, arthritis pain (e.g., osteoarthritis pain, rheumatoid arthritis pain), bursitis pain, pain from a gout attack, dental pain (e.g., toothache), surgical incision pain (e.g., tooth extraction), muscular aches, premenstrual and menstrual cramps, tendonitis, sore throat, acute trauma pain, and the like.

As used herein, pain relief and pain intensity are determined using clinical assessment scales known to those in the art. Examples of such assessments include, e.g., time-weighted sum of pain intensity difference (SPID), Pain-Intensity-Numerical Rating Scale (PI-NRS), pain intensity difference (PID), time-weighted total pain relief (TOTPAR), Pain Relief Numerical Rating Scale (PR-NRS). PI-NRS and PR-NRS are scales used to measure pain intensity and pain relief, whereas SPID and TOTPAR are assessments calculated using the data collected from the PI-NRS and PR-NRS to evaluate pain intensity or pain relief over a specified period of time.

Differences in pain intensity and/or pain relief can be assessed at baseline (at or just prior to dosage form administration) and compared with pain intensity and/or pain relief assessments at time points or time intervals post dosage form administration. For example, assessment can occur at about 10, 15, 20, 25, 30, 35, 40, 45, 50, 55, or 60 minutes post administration. In other aspects, assessment can occur at about 1.5, 2, 2.5, 3, 3.5, 4, 4.5, 5, 5.5, 6, 6.5, 7, 7.5, 8, 8.5, 9, 9.5, 10, 10.5, 11, 11.5, 12, 13, 14, 15, 16, 17, 18, 19, 20, 21, 22, 23, or 24 hours post administration.

The pharmaceutical dosage forms of the disclosure comprise acetaminophen in admixture with a salt of naproxen, e.g., naproxen sodium. Examples of various forms of acetaminophen are disclosed in WO1998027931. In preferred aspects, the pharmaceutical dosage forms include the acetaminophen and the naproxen sodium in the same granulation product. In some aspects, the pharmaceutical dosage forms include the acetaminophen and the naproxen sodium in a single granulation product.

In some aspects, the pharmaceutical dosage forms and in the form of one or more tablets and include the acetaminophen and the naproxen sodium in a single layer of the one or more tablets. These dosage forms are to the exclusion of multilayer, e.g., bilayer, tablets wherein the active ingredients are not in admixture with each other. For example, in a multilayer tablet including two or more active ingredient, each active ingredient is contained within its own layer, with minimal (or no) admixture of the actives.

Exemplary pharmaceutical dosage forms of the disclosure comprise acetaminophen in admixture with naproxen sodium. In these dosage forms, the weight ratio of the acetaminophen to the naproxen sodium is from about 2:1 to about 4:1, for example, about 2:1, 2.5:1, 3:1, 3.5:1, or 4:1. In one aspect the weight ratio of the acetaminophen to the naproxen sodium is about 3:1.

In some aspects of the disclosure, the pharmaceutical dosage forms comprise about 600 mg to about 700 mg of acetaminophen, for example, about 600, 625, 650, 675, or 700 mg of acetaminophen. In some aspects, the pharmaceutical dosage form comprises about 650 mg of acetaminophen.

In some aspects of the disclosure, the pharmaceutical dosage forms comprise about 150 mg to about 350 mg of naproxen sodium, for example, about 150, 160, 170, 180, 190, 200, 210, 220, 230, 240, 250, 260, 270, 280, 290, 300, 310, 320, 330, 340, or 350 mg of naproxen sodium. In some aspects, the pharmaceutical dosage form comprises about 220 mg of naproxen sodium.

In some aspects, the pharmaceutical dosage forms of the disclosure comprise about 650 mg of acetaminophen and about 220 mg of naproxen sodium, for example, 650 mg±10% of acetaminophen and 220 mg±10% of naproxen sodium. In other aspects, the pharmaceutical dosage forms of the disclosure comprise 650 mg±5% of acetaminophen and 220 mg±5% of naproxen sodium; 650 mg±4% of acetaminophen and 220 mg±4% of naproxen sodium; 650 mg±3% of acetaminophen and 220 mg±3% of naproxen sodium; 650 mg±2% of acetaminophen and 220 mg±2% of naproxen sodium; or 650 mg±1% of acetaminophen and 220 mg±1% of naproxen sodium.

Also within the scope of the disclosure are pharmaceutical dosage forms, for example, unit pharmaceutical dosage forms, that comprise about 325 mg of acetaminophen and about 110 mg of naproxen sodium, for example, 325 mg±10% of acetaminophen and 110 mg±10% of naproxen sodium. In other aspects, the pharmaceutical dosage forms of the disclosure comprise 325 mg±5% of acetaminophen and 110 mg±5% of naproxen sodium; 325 mg±4% of acetaminophen and 110 mg±4% of naproxen sodium; 325 mg±3% of acetaminophen and 110 mg±3% of naproxen sodium; 650 mg±2% of acetaminophen and 220 mg±2% of naproxen sodium; or 325 mg±1% of acetaminophen and 110 mg±1% of naproxen sodium.

The pharmaceutical dosage forms of the disclosure comprise a core tablet that includes an admixture of acetaminophen and naproxen sodium. The core tablets can also include from about 35 wt. % to about 45 wt. %, for example, about 35, 36, 37, 38, 39, 40, 41, 42, 43, 44, or 45 wt. % of acetaminophen. These core tablets can include from about 10 wt. % to about 15 wt. %, for example, about 10, 10.5, 11, 11.5, 12, 12.5, 13, 13.5, 14, 14.5, or 15 wt. % of naproxen sodium.

The core tablets of the disclosure will include other pharmaceutically acceptable excipients. The core tablets of the disclosure can include microcrystalline cellulose, for example, in an amount ranging from about 14 wt. % to about 18 wt. % of microcrystalline cellulose. In some aspects, the core tablets of the disclosure include about 14, 14.5, 15, 15.5, 16, 16.5, 17, 17.5, or 18 wt. % of microcrystalline cellulose.

The core tablets of the disclosure can include lactose, for example lactose monohydrate. In those core tablets comprising lactose monohydrate, the lactose monohydrate can be present in an amount ranging from about 4 wt. % to about 8 wt. % of lactose monohydrate. In some aspects, the core tablets of the disclosure include about 4, 4.5, 5, 5.5, 6, 6.5, 7, 7.5, or about 8 wt. % of lactose monohydrate.

The core tablets of the disclosure can include crospovidone. In those core tablets comprising crospovidone, the total amount of crospovidone in the core tablet can be in an amount of from about 3 wt. % to about 7 wt. % of crospovidone. In some aspects, the core tablets of the disclosure include about 3, 3.5, 4, 4.5, 5, 5.5, 6, 6.5, or 7 wt. % of crospovidone.

In some aspects, the core tablets of the disclosure comprise naproxen sodium, acetaminophen, microcrystalline cellulose, lactose monohydrate, and crospovidone. In some aspects, the core tablets comprise about 10 wt. % to about 15 wt. % of naproxen sodium; about 35 wt. % to about 45 wt. % of acetaminophen; about 14 wt. % to about 18 wt. % of microcrystalline cellulose; about 4 wt. % to about 8 wt. % lactose monohydrate; and 3 wt. % to about 7 wt. % crospovidone.

In other aspects, the core tablets consist essentially of naproxen sodium, acetaminophen, microcrystalline cellulose, lactose monohydrate, and crospovidone. In some aspects, the core tablets consist essentially of about 10 wt. % to about 15 wt. % of naproxen sodium; about 35 wt. % to about 45 wt. % of acetaminophen; about 14 wt. % to about 18 wt. % of microcrystalline cellulose; about 4 wt. % to about 8 wt. % lactose monohydrate; and 3 wt. % to about 7 wt. % crospovidone.

The core tablets of the disclosure can further include, in addition to naproxen sodium, acetaminophen, microcrystalline cellulose, lactose monohydrate, and crospovidone, starch, for example, pregelatinized starch. In those core tablets comprising pregelatinized starch, the pregelatinized starch can be present in an amount of from about 7 wt. % to about 12 wt. % of pregelatinized starch. In some aspects, the core tablets of the disclosure include about 7, 7.5, 8, 8.5, 9, 9.5, 10, 10.5, 11, 11.5, or 12 wt. % of pregelatinized starch.

The core tablets of the disclosure can further include silicified microcrystalline cellulose, in addition to naproxen sodium, acetaminophen, microcrystalline cellulose, lactose monohydrate, and crospovidone. In other aspects, the core tablets of the disclosure can further include silicified microcrystalline cellulose, in addition to naproxen sodium, acetaminophen, silicified microcrystalline cellulose, lactose monohydrate, crospovidone, and pregelatinized starch. In those core tablets comprising silicified microcrystalline cellulose, the silicified microcrystalline cellulose is present in an amount of from about 7 wt. % to about 12 wt. % of silicified microcrystalline cellulose. In some aspects, the core tablets of the disclosure include about 7, 7.5, 8, 8.5, 9, 9.5, 10, 10.5, 11, 11.5, or 12 wt. % of silicified microcrystalline cellulose.

The core tablets of the disclosure can further include magnesium stearate, in addition to naproxen sodium, acetaminophen, microcrystalline cellulose, lactose monohydrate, and crospovidone. In other aspects, the core tablets of the disclosure can further include magnesium stearate, in addition to naproxen sodium, acetaminophen, microcrystalline cellulose, lactose monohydrate, crospovidone, and pregelatinized starch. In other aspects, the core tablets of the disclosure can further include magnesium stearate, in addition to naproxen sodium, acetaminophen, microcrystalline cellulose, lactose monohydrate, crospovidone, pregelatinized starch, and silicified microcrystalline cellulose. In those core tablets comprising magnesium stearate, the magnesium stearate is present in an amount of from about 0.5 wt. % to about 1.5 wt. % of magnesium stearate. In some aspects, the core tablets of the disclosure include about 0.5, 0.6, 0.7, 0.8, 0.9, 1, 1.1, 1.2, 1.3, 1.4, or 1.5 wt. % of magnesium stearate.

In some aspects, the core tablets of the disclosure comprise about 10 wt. % to about 15 wt. % of naproxen sodium; about 35 wt. % to about 45 wt. % of acetaminophen; about 14 wt. % to about 18 wt. % of microcrystalline cellulose; about 4 wt. % to about 8 wt. % of lactose monohydrate; about 3 wt. % to about 7 wt. % of crospovidone; about 2 wt. % to about 5 wt. % of pregelatinized starch; about 7 wt. % to about 12 wt. % of silicified microcrystalline cellulose; and 0.5 wt. % to about 1.5 wt. % of magnesium stearate.

In some aspects, the core tablets of the disclosure consist essentially of about 10 wt. % to about 15 wt. % of naproxen sodium; about 35 wt. % to about 45 wt. % of acetaminophen; about 14 wt. % to about 18 wt. % of microcrystalline cellulose; about 4 wt. % to about 8 wt. % of lactose monohydrate; about 3 wt. % to about 7 wt. % of crospovidone; about 2 wt. % to about 5 wt. % of pregelatinized starch; about 7 wt. % to about 12 wt. % of silicified microcrystalline cellulose; and 0.5 wt. % to about 1.5 wt. % of magnesium stearate.

The pharmaceutical dosage forms of the disclosure may further comprise one or more active ingredients other than acetaminophen and naproxen. For example, pharmaceutical dosage forms of the disclosure may further comprise a decongestant (e.g., phenylephrine, pseudoephedrine), an antihistamine (e.g. cetirizine, diphenhydramine, fexofenadine, loratadine), a mucolytic (e.g., guaifenesin), a sleep aid (e.g., diphenhydramine, doxylamine, melatonine), an antitussive (e.g., dextromethorphan) or a mixture thereof.

According to the methods of the disclosure, the pharmaceutical dosage forms are administered as one or more tablets, for example, one or more film-coated tablets. In those aspects wherein the pharmaceutical dosage form is administered as one tablet, the tablet comprises about 600 mg to about 700 mg of acetaminophen and about 150 mg to about 250 mg of naproxen sodium, for example, 650 mg of acetaminophen and 220 mg of naproxen sodium.

In those aspects wherein the pharmaceutical dosage form is administered as two tablets, each tablet comprises about 300 mg to about 350 mg of acetaminophen and about 75 mg to about 125 mg of naproxen sodium. In some of these aspects, each tablet comprises about 325 mg of acetaminophen and about 110 mg of naproxen.

In some methods of the disclosure, the pharmaceutical dosage forms are administered once per day to induce minor pain relief in a person in need thereof. In other aspects, the pharmaceutical dosage forms are administered twice per day, to induce minor pain relief in a person in need thereof. In some aspects, the pharmaceutical dosage forms are administered every six hours, to induce minor pain relief in a person in need thereof. In some aspects, the pharmaceutical dosage forms are administered every eight hours, to induce minor pain relief in a person in need thereof. In some aspects, the pharmaceutical dosage forms are administered every 12 hours, to induce minor pain relief in a person in need thereof. In some aspects, the pharmaceutical dosage forms are administered every 24 hours, to induce minor pain relief in a person in need thereof.

In some methods of the disclosure, the pharmaceutical dosage forms are administered at time intervals of about six hours. In some methods of the disclosure, the pharmaceutical dosage forms are administered at time intervals of about eight hours. In some methods of the disclosure, the pharmaceutical dosage forms are administered at time intervals of about 12 hours. In some methods of the disclosure, the pharmaceutical dosage forms are administered at variable time intervals including six hours, eight hours, 12 hours, or a combination thereof. In some methods of the disclosure, the pharmaceutical dosage forms are administered at variable time intervals including six hours, eight hours, 12 hours, 24 hours, or a combination thereof.

Those of ordinary skill in the art can prepare core tablets described herein using granulation methods. In some aspects, a granulation product, for example, an intragranular portion, is prepared by granulating naproxen sodium, acetaminophen, microcrystalline cellulose, lactose monohydrate and crospovidone with a binder solution comprising purified water and pregelatinized starch. The granulation product can be combined with silicified microcrystalline cellulose, additional crospovidone, and magnesium stearate to form a product blend. The product blend can be compressed into a core tablet using tablet compression methods in the art. The resulting core tablets can be coated, for example, film coated, using methods of the art.

In some aspects, the granulation product, for example, an intragranular portion, is prepared by granulating naproxen sodium (about 10 wt. % to about 15 wt. %), acetaminophen (about 35 wt. % to about 45 wt. %), microcrystalline cellulose (about 14 wt. % to about 18 wt. %), lactose monohydrate (about 4 wt. % to about 8 wt. %) and crospovidone (about 0.5 wt. % to about 2.5 wt. %) with a binder solution, for example, a binder spray, comprising purified water and pregelatinized starch (about 7 wt. % to about 12 wt. %). Referenced weight percentages for preparing the granulation product are to the exclusion of the purified water.

In some aspects, the weight ratio of acetaminophen to naproxen sodium in the intragranular portion is from about 2:1 to about 4:1, for example, about 2:1, 2.5:1, 3:1, 3.5:1, or 4:1. In one aspect the weight ratio of the acetaminophen to the naproxen sodium in the intragranular portion is about 3:1.

Preferably, the acetaminophen, naproxen sodium, microcrystalline cellulose, lactose monohydrate, and crospovidone are sifted prior to combination with the binder solution. In some aspects, the granulation product is screened, milled, re-screened (e.g., to about 0.062″), and blended using techniques in the art.

In some aspects, the granulation product is combined with silicified microcrystalline cellulose (about 7 wt. % to about 12 wt. %) and additional crospovidone (additional about 2 wt. % to about 5 wt. %) in an extra-granulation process. Magnesium stearate (0.5 wt. % to about 1.5 wt. %) can also be added and blended in the extra-granulation process. The extra-granulation process produces a product blend. The product blend can be compressed into one or more core tablets. Administration of one or more core tablets to a human will induce minor pain relief. The one or more core tablets can be film-coated, using methods of the art. Administration of one or more film-coated tablets to a human will induce minor pain relief.

In some aspects, the intragranular portion is combined with an extragranular portion that comprises a binder, a lubricant, a superdisintegrant, or a combination thereof. In some aspects, the intragranular portion is combined with an extragranular portion that comprises a binder. In some aspects, the intragranular portion is combined with an extragranular portion that comprises a lubricant. In some aspects, the intragranular portion is combined with an extragranular portion that comprises a superdisintegrant. In some aspects, the intragranular portion is combined with an extragranular portion that comprises a binder and a lubricant. In some aspects, the intragranular portion is combined with an extragranular portion that comprises a lubricant and a superdisintegrant. In some aspects, the intragranular portion is combined with an extragranular portion that comprises a binder and a superdisintegrant. In some aspects, the intragranular portion is combined with an extragranular portion that comprises a binder, a lubricant, and a superdisintegrant. An exemplary binder is silicified microcrystalline cellulose. An exemplary lubricant is magnesium stearate. An exemplary superdisintegrant is crospovidone.

The combination of intragranular portion and extragranular portion can be compressed into one or more core tablets. The one or more core tablets can be film-coated, using methods of the art. Administration of one or more core tablets to a human will induce minor pain relief. Administration of one or more film-coated tablets to a human will induce minor pain relief.

Acetaminophen and naproxen each include reactive functional groups, for example, —OH, —COOH, —NHC(O)CH₃. It is noteworthy that little to no acetaminophen-naproxen by-products have been observed in the core tablets that are produced according to the methods described herein.

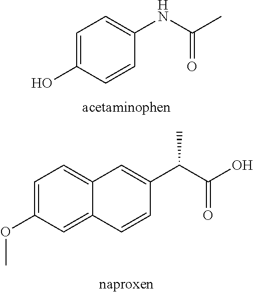 [SMILES: CC(=O)Nc1ccc(O)cc1 ; COc1ccc2cc([C@H](C)C(=O)O)ccc2c1]

The pharmaceutical dosage forms of the disclosure can be provided as part of an OTC pharmaceutical pack, for example, a blister pack. In other aspects, the pharmaceutical dosage forms of the disclosure are provided in OTC bottles including, for example, 10, 20, 30, 40, 50, 60, 70, 80, 90, 100, 150, 200, 250, 300, 350, 400, 450, or 500 units of the pharmaceutical dosage form.

Surprisingly, the pharmaceutical dosage forms have been demonstrated to more quickly induce pain relief and/or to more quickly reduce pain intensity in humans experience minor pain.

The pharmaceutical dosage forms of the disclosure have been shown to provide greater pain relief, in a shorter amount of time, as compared to placebo, as measured by Pain Relief Scores. For example, according to the disclosed methods, the Pain Relief Score of a human in need of minor pain relief assessed at 15 minutes post-administration of a described pharmaceutical dosage form (e.g., comprising about 650 mg of acetaminophen in admixture with about 220 mg of naproxen sodium) is greater, as compared to a Pain Relief Score of a human in need of minor pain relief at 15 minutes post-administration of a placebo.

The pharmaceutical dosage forms of the disclosure have been shown to provide greater pain relief, in a shorter amount of time, as compared to a dosage form comprising 1000 mg of acetaminophen and 440 mg of naproxen sodium, as measured by Pain Relief Scores. For example, according to the disclosed methods, the Pain Relief Score of a human in need of minor pain relief assessed at 15 minutes post-administration of a described pharmaceutical dosage form (e.g., comprising about 650 mg of acetaminophen in admixture with about 220 mg of naproxen sodium) is greater, as compared to a Pain Relief Score of a human in need of minor pain relief at 15 minutes post-administration of a pharmaceutical dosage form not of the disclosure (e.g., comprising about 1000 mg of acetaminophen and about 440 mg of naproxen sodium).

The greater pain relief effect of the described dosage forms over those dosage forms comprising about 1000 mg of acetaminophen and about 440 mg of naproxen sodium is also observed when measured by Pain Intensity Difference Scores. According to the disclosed methods, the Pain Intensity Difference Score of a human in need of minor pain relief assessed at 15 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief at 15 minutes post-administration of a pharmaceutical dosage form not of the disclosure (e.g., comprising about 1000 mg of acetaminophen and about 440 mg of naproxen sodium). According to the disclosed methods, the Pain Intensity Difference Score of a human of minor pain relief assessed at 30 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief at 30 minutes post-administration of a pharmaceutical dosage form not of the disclosure (e.g., comprising about 1000 mg of acetaminophen and about 440 mg of naproxen sodium). According to the disclosed methods, the Pain Intensity Difference Score of a human of minor pain relief assessed at 45 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief at 45 minutes post-administration of a pharmaceutical dosage form not of the disclosure (e.g., comprising about 1000 mg of acetaminophen and about 440 mg of naproxen sodium). According to the disclosed methods, the Pain Intensity Difference Score of a human of minor pain relief assessed at 60 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief at 60 minutes post-administration of a pharmaceutical dosage form not of the disclosure (e.g., comprising about 1000 mg of acetaminophen and about 440 mg of naproxen sodium).

The pharmaceutical dosage forms of the disclosure have unexpectedly been shown to provide greater minor pain relief, in a shorter amount of time, as compared to a dosage form comprising about 440 mg of naproxen sodium in the absence of acetaminophen, as measured by Pain Relief Scores. For example, according to the disclosed methods, the Pain Relief Score of a human in need of minor pain relief, assessed at 15 minutes post-administration of a described pharmaceutical dosage form (e.g., comprising about 650 mg of acetaminophen in admixture with about 220 mg of naproxen sodium) is greater, as compared to a Pain Relief Score of a human in need of minor pain relief at 15 minutes post-administration of a pharmaceutical dosage form not of the disclosure (e.g., comprising about 440 mg of naproxen sodium in the absence of acetaminophen. According to the disclosed methods, the Pain Relief Score of a human in need of minor pain relief, assessed at 30 minutes post-administration of a described pharmaceutical dosage form (e.g., comprising about 650 mg of acetaminophen in admixture with about 220 mg of naproxen sodium) is greater, as compared to a Pain Relief Score of a human in need of minor pain relief at 30 minutes post-administration of a pharmaceutical dosage form not of the disclosure (e.g., comprising about 440 mg of naproxen sodium in the absence of acetaminophen. According to the disclosed methods, the Pain Relief Score of a human in need of minor pain relief, assessed at 45 minutes post-administration of a described pharmaceutical dosage form (e.g., comprising about 650 mg of acetaminophen in admixture with about 220 mg of naproxen sodium) is greater, as compared to a Pain Relief Score of a human in need of minor pain relief at 45 minutes post-administration of a pharmaceutical dosage form not of the disclosure (e.g., comprising about 440 mg of naproxen sodium in the absence of acetaminophen. According to the disclosed methods, the Pain Relief Score of a human in need of minor pain relief, assessed at 60 minutes post-administration of a described pharmaceutical dosage form (e.g., comprising about 650 mg of acetaminophen in admixture with about 220 mg of naproxen sodium) is greater, as compared to a Pain Relief Score of a human in need of minor pain relief at 60 minutes post-administration of a pharmaceutical dosage form not of the disclosure (e.g., comprising about 440 mg of naproxen sodium in the absence of acetaminophen.

The greater minor pain relief effect of the described dosage forms over those dosage forms comprising about 440 mg of naproxen sodium in the absence of acetaminophen is also observed when measured by Pain Intensity Difference Scores. According to the disclosed methods, the Pain Intensity Difference Score of a human in need of minor pain relief, assessed at 15 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief, at 15 minutes post-administration of a pharmaceutical dosage form not of the disclosure (e.g., comprising about 440 mg of naproxen sodium in the absence of acetaminophen). According to the disclosed methods, the Pain Intensity Difference Score of a human in need of minor pain relief, assessed at 30 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief, at 30 minutes post-administration of a pharmaceutical dosage form not of the disclosure (e.g., comprising about 440 mg of naproxen sodium in the absence of acetaminophen). According to the disclosed methods, the Pain Intensity Difference Score of a human in need of minor pain relief, assessed at 45 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief, at 45 minutes post-administration of a pharmaceutical dosage form not of the disclosure (e.g., comprising about 440 mg of naproxen sodium in the absence of acetaminophen). According to the disclosed methods, the Pain Intensity Difference Score of a human in need of minor pain relief, assessed at 60 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief, at 60 minutes post-administration of a pharmaceutical dosage form not of the disclosure (e.g., comprising about 440 mg of naproxen sodium in the absence of acetaminophen).

Also surprisingly, the pharmaceutical dosage forms of the disclosure have been shown to provide greater minor pain relief, in a shorter amount of time, compared to a dosage form comprising about 650 mg of acetaminophen and about 10 mg of hydrocodone, as measured by Pain Relief Scores. For example, surprisingly, according to the disclosed methods, the Pain Relief Score of a human in need of minor pain relief, assessed at 15 minutes post-administration of a described pharmaceutical dosage form (e.g., comprising about 650 mg of acetaminophen in admixture with about 220 mg of naproxen sodium) is greater, as compared to a Pain Relief Score of a human in need of minor pain relief at 15 minutes post-administration of a pharmaceutical dosage form not of the disclosure (e.g., comprising about 650 mg of acetaminophen and about 10 mg of hydrocodone).

The greater minor pain relief effect of the described dosage forms over those dosage forms comprising about 650 mg of acetaminophen and about 10 mg of hydrocodone is also observed when measured by Pain Intensity Difference Scores. According to the disclosed methods, the Pain Intensity Difference Score of a human in need of minor pain relief, assessed at 15 minutes post-administration of a described pharmaceutical dosage form (e.g., comprising about 650 mg of acetaminophen in admixture with about 220 mg of naproxen sodium) is greater, as compared to a Pain Intensity Difference Score of a human in need of minor pain relief, at 15 minutes post-administration of a pharmaceutical dosage form not of the disclosure (e.g., comprising about 650 mg of acetaminophen and about 10 mg of hydrocodone).

Also according to the described methods, the Time-Weighted Sum of Pain Intensity Difference Score from baseline to six hours is greater in a human in need of minor pain relief administered a described pharmaceutical dosage form (e.g., comprising about 650 mg of acetaminophen in admixture with about 220 mg of naproxen sodium), as compared to a Time-Weighted Sum of Pain Intensity Difference Score from Baseline to six hours of a human in need of minor pain relief administered a pharmaceutical dosage form not of the disclosure (e.g., comprising about 650 mg of acetaminophen and about 10 mg of hydrocodone).

Also according to the described methods, the Time-Weighted Sum of Pain Intensity Difference Score from baseline to eight hours is greater in a human in need of minor pain relief administered a described pharmaceutical dosage form (e.g., comprising about 650 mg of acetaminophen in admixture with about 220 mg of naproxen sodium), as compared to a Time-Weighted Sum of Pain Intensity Difference Score from Baseline to eight hours of a human in need of minor pain relief administered a pharmaceutical dosage form not of the disclosure (e.g., comprising about 650 mg of acetaminophen and about 10 mg of hydrocodone).

Also according to the described methods, the Time-Weighted Sum of Pain Intensity Difference Score from baseline to 12 hours is greater in a human in need of minor pain relief administered a described pharmaceutical dosage form (e.g., comprising about 650 mg of acetaminophen in admixture with about 220 mg of naproxen sodium), as compared to a Time-Weighted Sum of Pain Intensity Difference Score from Baseline to 12 hours of a human in need of minor pain relief administered a pharmaceutical dosage form not of the disclosure (e.g. comprising about 650 mg of acetaminophen and about 10 mg of hydrocodone).

Also according to the described methods, the Time-Weighted Sum of Total Pain Relief Score from baseline to six hours is greater in a human in need of minor pain relief administered a described pharmaceutical dosage form (e.g., comprising about 650 mg of acetaminophen in admixture with about 220 mg of naproxen sodium), as compared to a Time-Weighted Sum of Total Pain Relief Score from Baseline to six hours of a human in need of minor pain relief administered a pharmaceutical dosage form not of the disclosure (e.g. comprising about 650 mg of acetaminophen and about 10 mg of hydrocodone).

Also according to the described methods, the Time-Weighted Sum of Total Pain Relief Score from baseline to eight hours is greater in a human in need of minor pain relief administered a described pharmaceutical dosage form (e.g., comprising about 650 mg of acetaminophen in admixture with about 220 mg of naproxen sodium), as compared to a Time-Weighted Sum of Total Pain Relief Score from Baseline to eight hours of a human in need of minor pain relief administered a pharmaceutical dosage form not of the disclosure (e.g., comprising about 650 mg of acetaminophen and about 10 mg of hydrocodone).

Also according to the described methods, the Time-Weighted Sum of Total Pain Relief Score from baseline to 12 hours is greater in a human in need of minor pain relief administered a described pharmaceutical dosage form (e.g., comprising about 650 mg of acetaminophen in admixture with about 220 mg of naproxen sodium), as compared to a Time-Weighted Sum of Total Pain Relief Score from Baseline to 12 hours of a human in need of minor pain relief administered a pharmaceutical dosage form not of the disclosure (e.g., comprising about 650 mg of acetaminophen and about 10 mg of hydrocodone).

### EXAMPLES

### Example 1

### Granulation

| | | | |
|---|---|---|---|
| | Batch | Unit Weight | Weight |
| Description | Wt. (g) | (mg) | Percent |

Granulation Raw Materials

| | | | |
|---|---|---|---|
| Naproxen Sodium, USP | 2,200 | 110.00 | 13.74 |
| Acetaminophen, USP | 6,500 | 325.00 | 40.59 |
| Microcrystalline Cellulose, NF | 2,600 | 130.00 | 16.24 |
| Lactose Monohydrate, NF, Ph. Eur. | 1,050 | 52.50 | 6.56 |
| Impalpable | | | |
| Crospovidone, NF Type A | 300 | 15.00 | 1.87 |

Binder Solution (6.0% Pregelatinized Starch Slurry)

| | | | |
|---|---|---|---|
| Pregelatinized Starch, NF | 600 | 30.00 | 3.75 |
| Purified Water, USP | 9,400 | Qs | N/A |

Spray Flush

| | | | |
|---|---|---|---|
| Purified Water, USP | 1,500 | Qs | N/A |
| Total: | 13,250 | — | — |

Binder spray is prepared by combining purified water and pregelatinized starch in a high shear propeller mixer for about 30 minutes at ambient temperature, or until the suspension is visually uniform and free of clumps. Pre-sifted acetaminophen, naproxen sodium, microcrystalline cellulose, lactose monohydrate, and crospovidone combined and the mixture granulated with the binder spray. The resulting granulations is screened, milled, re-screened (0.062″), and blended.

### Example 2

### Extra-Granulation

Extra-Granulation Materials

| | | Unit | |
|---|---|---|---|
| | Batch | Weight | Weight |
| Description | Wt. (g) | (mg) | Percent |
| Product Granulation from Example 1 | 13,250 | 662.50 | 82.74 |
| Silicified Microcrystalline Cellulose, NF | 1,500 | 75.00 | 9.37 |
| Crospovidone, NF Type A | 800 | 40.00 | 5.00 |
| Magnesium Stearate, NF 2257 Vegetable | 150 | 7.50 | 0.94 |
| Source | | | |
| Total: | 15,700 | — | 98.04 |

The granulation of Example 1 is combined with silicifed microcrystalline cellulose and crospovidone and blended for about 30 minutes. Magnesium stearate added and the mixture blended for about an additional 5 minutes.

### Example 3

### Core Tablets

Core tablets prepared via compression of the product blend of Example 2

Core Tablets

| | Batch | Unit Weight | Weight |
|---|---|---|---|
| Description | Wt. (g) | (mg) | Percent |
| Product Blend from Example 2 | 15,700 | 785.00 | 98.04 |
| Core Tablets: | 15,700 | 785.00 | — |

Core tablets preferably have the following characteristics:

| Parameter | Target | | Range | |
|---|---|---|---|---|
| Average Tablet Weight | 0.785 | | 0.462 g-0.809 g | |
| Average Tablet Thickness | 6.4 | mm | 6.2 mm-6.8 mm | |
| Average Tablet Hardness | 16.0 | kP | 10.0-18.0 | kP |
| Friability | 1% | | ≤1% | |
| Disintegration | 15 | minutes | ≤15 | minutes |

### Example 4

### Coated Tablets

The core tablets of Example 3 are spray-coated with a coating solution (11% w/w solids).

| | | | |
|---|---|---|---|
| | Batch | Unit Weight | Weight |
| Description | Wt. (g) | (mg) | Percent |

Coated Tablets Materials

| | | | |
|---|---|---|---|
| Core Tablets from Example 3 | 13,000 | 785.00 | 98.04 |

Coating Solution (11% w/w solids)

| | | | |
|---|---|---|---|
| Opadry ™ White (Colorcon, Inc.) | 374 | 15.70 | 1.96 |
| Purified Water, USP | 3,026 | — | — |
| Coated Tablets: | — | 800.70 | 100.00 |

Coated tablets comprising 325 mg of acetaminophen and 110 mg of naproxen sodium are prepared according to these methods.

### Example 5

### Dissolution

The dissolution of coated tablets prepared as described herein was analyzed using the Paddle method (USP Apparatus II) set at 50 RPM. Samples were analyzed using an HPLC equipped with a YMC Hydrosphere C18, 50 mm×4.6 mm 2 μm particle size column, a UV detector at 280 nm and switching to 332 nm at 2.5 min, with a flow rate of 1.0ml/min and an injection volume of 10 μL. Mobile Phase A; 40 mM ammonium acetate/0.1% TFA in water. Mobile Phase B: 40 mM ammonium acetate/0.1% TFA in [methanol: water: acetonitrile (75:15:10). Dissolution Media was 0.1M sodium phosphate buffer at pH 7.4. The dissolution criteria is Q=80 (% released) at 45 minutes for acetaminophen (APAP) and a Q=80 (% released) at 45 minutes for naproxen.

### Example 6

### Stability

Stability tests performed at 25° C., 60% relative humidity and 40° C., 75% relative humidity were performed on coated tablets prepared as described herein. Data after one month and after three months indicated good stability at both temperature/relative humidity conditions.

### Example 7

### Phase 2 Clinical Trial and Results: NCT03879408

This is a randomized, double-blind, placebo-and active-controlled, parallel-group study to evaluate the analgesic efficacy and safety of concomitantly administered naproxen sodium 440 mg with acetaminophen 1000 mg and concomitantly administered naproxen sodium 220 mg with acetaminophen 650 mg, compared with a fixed combination of hydrocodone 10 mg/acetaminophen 650 mg, naproxen sodium 440 mg, and placebo over a twelve-hour period after surgical extraction of four third molars.

### Primary Outcome Measures

1. Time-Weighted Sum of Pain Intensity Difference Score From Baseline (0 Hour) to 6 Hours (SPID 0-6) [Time Frame: Baseline (0 hour) up to 6 hours post-dose]. Time-weighted sum of the pain intensity difference (SPID) score was measured using a Pain Intensity-Numerical Rating Scale (PI-NRS) ranging from 0-10 (0=no pain, 10=very severe pain). The possible range of SPID for 0-6 hours was from −60 to 60. A higher value of SPID indicated greater pain relief. PID was the difference between baseline pain intensity and pain intensity at assessment. Time-weighted sum of the pain intensity difference scores were derived by first multiplying each PID score by the time from the previous time point and adding these time-weighted PID scores together over the intervals from 0 to 6 hours.

2. Time-weighted Sum of Pain Intensity Difference Score From Baseline (0 Hour) to 12 Hours (SPID 0-12) [Time Frame: Baseline (0 hour) up to 12 hours post-dose]. Time-weighted sum of the pain intensity difference score was measured using a PI-NRS ranging from 0-10 (0=no pain, 10=very severe pain). The possible range of SPID for 0-12 hours was from-120 to 120. A higher value of SPID indicated greater pain relief. PID was the difference between baseline pain intensity and pain intensity at assessment (12 hours). Time-weighted sum of the pain intensity difference scores were derived by first multiplying each PID score by the time from the previous time point and adding these time-weighted PID scores together over the intervals from 0 to 12 hours.

### Secondary Outcome Measures

1. Time-Weighted Sum of Total Pain Relief Score From Baseline (0 Hour) to 6 Hours (TOTPAR 0-6) [Time Frame: Baseline (0 hour) up to 6 hours post-dose]. Time-weighted total pain relief (TOTPAR) was measured using a Pain Relief Numerical Rating Scale (PR-NRS) ranging from 0-10 (0=no relief, 10=complete relief). Total pain relief scores (TOTPARs) were calculated by multiplying the pain relief score at each postdose time point by the duration (in hours) since the preceding time point and then summing these values. The minimum value was 0, and the maximum value was 60. Higher scores was indicative of more pain relief.

2. Time-Weighted Sum of Total Pain Relief Score From Baseline (0 Hour) to 8 Hours (TOTPAR 0-8) [Time Frame: Baseline (0 hour) up to 8 hours post-dose]. TOTPAR was measured using a PR-NRS ranging from 0-10 (0=no relief, 10=complete relief). Total pain relief scores (TOTPARs) were calculated by multiplying the pain relief score at each postdose time point by the duration (in hours) since the preceding time point and then summing these values. The minimum value was 0, and the maximum value was 80. Higher scores was indicative of more pain relief.

3. Time-Weighted Sum of Total Pain Relief Score From Baseline (0 Hour) to 12 Hours (TOTPAR 0-12) [Time Frame: Baseline (0 hour) up to 12 hours post-dose]. TOTPAR was measured using a PR-NRS ranging from 0-10 (0=no relief, 10=complete relief). Total pain relief scores (TOTPARs) were calculated by multiplying the pain relief score at each postdose time point by the duration (in hours) since the preceding time point and then summing these values. The minimum value was 0, and the maximum value was 120. Higher scores was indicative of more pain relief.

4. Time-Weighted Sum of Pain Intensity Difference Score From Baseline (0 Hour) to 8 Hours (SPID 0-8) [Time Frame: Baseline (0 hour) up to 8 hours post-dose]. Time-weighted sum of the pain intensity difference score was measured using a PI-NRS ranging from 0-10 (0=no pain, 10=very severe pain). The possible range of SPID for 0-8 hours was from −80 to 80. A higher value of SPID indicated greater pain relief. PID was the difference between baseline pain intensity and pain intensity at assessment (8 hours). Time-weighted sum of the pain intensity difference scores were derived by first multiplying each PID score by the time from the previous time point and adding these time-weighted PID scores together over the intervals from 0 to 8 hours.

5. Pain Relief (PAR) Scores at Individual Timepoints [Time Frame: 0.25 hours, 0.5 hours, 0.75 hours, 1 hours, 1.25 hours, 1.5 hours, 2 hours, 3 hours, 4 hours, 5 hours, 6 hours, 7 hours, 8 hours, 9 hours, 10 hours, 11 hours, and 12 hours]. Participants answered a question at individual time points: “how much relief do you have from your starting pain?” on a 11-point PR-NRS. Scale ranged from 0=no relief to 10=complete relief. Higher score indicated improvement in pain.

6. Pain Intensity Difference (PID) Scores at Individual Time Points [Time Frame: 0.25 hours, 0.5 hours, 0.75 hours, 1 hours, 1.25 hours, 1.5 hours, 2 hours, 3 hours, 4 hours, 5 hours, 6 hours, 7 hours, 8 hours, 9 hours, 10 hours, 11 hours, and 12 hours]. Pain Intensity was self-reported over 12 hours, using a pain rating of 0-10 on the PI-NRS, with score ranged from 0-10 (0=no pain; 10 =worst imaginable pain). Pain intensity differences were calculated with respect to baseline at each time point after study drug administration.

7. Participant's Global Evaluation of Study Medication OR Overall Impression of Study Medication According to Participant's Global Evaluation [Time Frame: Up to 12 hours]. Participants were asked to rate their overall impression of the study medication using the following scale: poor (0), fair (1), good (2), very good (3), and excellent (4) where higher score represented better outcome.

### Inclusion Criteria:

1. Males or females 17-50 years old
2. Weigh 100 pounds or greater and have a body mass index (BMI) of 18.5 to 35 (inclusive) at screening
3. Surgical removal of up to four third molars, of which, two must be mandibular impactions
4. Meets requirements for post-surgical pain level
5. Females of childbearing potential and males agree to contraceptive requirements of study
6. Have a negative urine drug screen at screening, and on day of surgical procedure

### Exclusion Criteria:

1. Pregnant female, breastfeeding, trying to become pregnant or male with pregnant partner or partner currently trying to become pregnant
2. Have known allergy or hypersensitivity to naproxen or other NSAIDS, including aspirin, or to acetaminophen, tramadol, hydrocodone or other opioids
3. Not able to swallow large tablets or capsules
4. History of any condition(s) in investigator's opinion, may jeopardize subject safety, well-being and integrity of study
5. Use analgesics 5 or more times per week
6. History of chronic tranquilizer use, heavy drinking, substance abuse as judged by investigator site staff within last 5 years
7. Use of immunosuppressive drugs within 2 weeks of screening
8. History of peptic ulcer disease or gastrointestinal bleeding in the last two years or hematologic bleeding

| | Arm/Group Title | | | | |
|---|---|---|---|---|---|
| | | | Arm 3: Naproxen | Arm 4: Naproxen | Arm 5: Hydrocodone |
| | | | Sodium 220 mg + | Sodium 440 mg + | 10 mg + |
| | Arm 1: | Arm 2: Naproxen | Acetaminophen | Acetaminophen | Acetaminophen |
| | Placebo | Sodium 440 mg | 650 mg | 1000 mg | 650 mg |
| Arm/Group | Participants | Participants | Participants | Participants | Participants |
| Description | received a | received a single | received a single | received a single | received a single |
| | single oral | oral dose of | oral dose of | oral dose of | oral dose of |
| | dose of four | naproxen sodium | naproxen sodium | naproxen sodium | hydrocodone 10 |
| | placebo | 440 milligrams | 220 mg tablet | 440 mg | mg (administered |
| | tablets at | (mg)(administered | (administered as | (administered as | as two |
| | baseline | as two naproxen | one naproxen | two naproxen | hydrocodone 5 |
| | (Day of | sodium 220 mg | sodium 220 mg | sodium 220 mg | mg tablets) and |
| | surgery- | tablets) and two | tablet) and | tablets) and | acetaminophen |
| | Day 1). | placebo tablets at | acetaminophen | acetaminophen | 650 mg (two |
| | | baseline (Day of | 650 mg (two | 1000 mg (two | acetaminophen |
| | | surgery-Day 1). | acetaminophen | acetaminophen | 325 mg tablets) |
| | | | 325 mg tablets) | 500 mg tablets) | and two placebo |
| | | | and one placebo | at baseline (Day | tablets at |
| | | | tablet at baseline | of surgery-Day | baseline (Day of |
| | | | (Day of surgery- | 1). | surgery-Day 1). |
| | | | Day 1). | | |

Overall Study

| | | | | | |
|---|---|---|---|---|---|
| Started | 35 | 69 | 61 | 63 | 62 |
| Completed | 33 | 65 | 58 | 62 | 61 |
| Not | 2 | 4 | 3 | 1 | 1 |
| Completed | | | | | |

Time-Weighted Sum of Pain Intensity Difference Score From Baseline (0 Hour) to 6 Hours (SPID 0-6): Time-weighted sum of the pain intensity difference (SPID) score was measured using a Pain Intensity-Numerical Rating Scale (PI-NRS) ranging from 0-10 (0=no pain, 10=very severe pain). The possible range of SPID for 0-6 hours was from −60 to 60. A higher value of SPID indicated greater pain relief. PID was the difference between baseline pain intensity and pain intensity at assessment. Time-weighted sum of the pain intensity difference scores were derived by first multiplying each PID score by the time from the previous time point and adding these time-weighted PID scores together over the intervals from 0 to 6 hours.

| | Arm/Group Title | | | | |
|---|---|---|---|---|---|
| | | | Arm 3: Naproxen | Arm 4: Naproxen | Arm 5: Hydrocodone |
| | | | Sodium 220 mg + | Sodium 440 mg + | 10 mg + |
| | Arm 1: | Arm 2: Naproxen | Acetaminophen | Acetaminophen | Acetaminophen |
| | Placebo | Sodium 440 mg | 650 mg | 1000 mg | 650 mg |
| Least | 2.36 (2.181) | 25.59 (1.562) | 30.28 (1.677) | 33.77 (1.633) | 23.60 (1.651) |
| Squares | | | | | |
| Mean | | | | | |
| (Standard | | | | | |
| Error) | | | | | |

Time-weighted Sum of Pain Intensity Difference Score From Baseline (0 Hour) to 12 Hours (SPID 0-12): Time-weighted sum of the pain intensity difference score was measured using a PI-NRS ranging from 0-10 (0=no pain, 10=very severe pain). The possible range of SPID for 0-12 hours was from-120 to 120. A higher value of SPID indicated greater pain relief. PID was the difference between baseline pain intensity and pain intensity at assessment (12 hours). Time-weighted sum of the pain intensity difference scores were derived by first multiplying each PID score by the time from the previous time point and adding these time-weighted PID scores together over the intervals from 0 to 12 hours.

| | Arm/Group Title | | | | |
|---|---|---|---|---|---|
| | | | | | Arm 5: |
| | | Arm 2: | Arm 3: Naproxen | Arm 4: Naproxen | Hydrocodone 10 |
| | | Naproxen | Sodium 220 mg + | Sodium 440 mg + | mg + |
| | Arm 1: | Sodium 440 | Acetaminophen | Acetaminophen | Acetaminophen |
| | Placebo | mg | 650 mg | 1000 mg | 650 mg |
| Least Squares | 5.80 (4.666) | 50.07 (3.341) | 55.47 (3.587) | 66.43 (3.494) | 39.94 (3.533) |
| Mean | | | | | |
| (Standard | | | | | |
| Error) | | | | | |

Time-Weighted Sum of Total Pain Relief Score From Baseline (0 Hour) to 6 Hours (TOTPAR 0-6): Time-weighted total pain relief (TOTPAR) was measured using a Pain Relief Numerical Rating Scale (PR-NRS) ranging from 0-10 (0=no relief, 10=complete relief). Total pain relief scores (TOTPARs) were calculated by multiplying the pain relief score at each postdose time point by the duration (in hours) since the preceding time point and then summing these values. The minimum value was 0, and the maximum value was 60. Higher scores was indicative of more pain relief.

| | Arm/Group Title | | | | |
|---|---|---|---|---|---|
| | | | | | Arm 5: |
| | | Arm 2: | Arm 3: Naproxen | Arm 4: Naproxen | Hydrocodone |
| | | Naproxen | Sodium 220 mg + | Sodium 440 mg + | 10 mg + |
| | Arm 1: | Sodium 440 | Acetaminophen | Acetaminophen | Acetaminophen |
| | Placebo | mg | 650 mg | 1000 mg | 650 mg |
| Least | 7.96 (2.581) | 35.20 (1.848) | 40.94 (1.984) | 45.13 (1.933) | 32.88 (1.954) |
| Squares | | | | | |
| Mean | | | | | |
| (Standard | | | | | |
| Error) | | | | | |

Time-Weighted Sum of Total Pain Relief Score From baseline (0 Hour) to 8 Hours (TOTPAR 0-8): TOTPAR was measured using a PR-NRS ranging from 0-10 (0=no relief, 10=complete relief). Total pain relief scores (TOTPARs) were calculated by multiplying the pain relief score at each postdose time point by the duration (in hours) since the preceding time point and then summing these values. The minimum value was 0, and the maximum value was 80. Higher scores was indicative of more pain relief.

| | Arm/Group Title | | | | |
|---|---|---|---|---|---|
| | | | | | Arm 5: |
| | | Arm 2: | Arm 3: Naproxen | Arm 4: Naproxen | Hydrocodone |
| | | Naproxen | Sodium 220 mg + | Sodium 440 mg + | 10 mg + |
| | Arm 1: | Sodium 440 | Acetaminophen | Acetaminophen | Acetaminophen |
| | Placebo | mg | 650 mg | 1000 mg | 650 mg |
| Least Squares | 11.12 | 47.19 | 54.37 (2.732) | 60.78 (2.662) | 41.61 (2.691) |
| Mean | (3.554) | (2.545) | | | |
| (Standard | | | | | |
| Error) | | | | | |

Time-Weighted Sum of Total Pain Relief Score From Baseline (0 Hour to 12 Hours (TOTPAR 0-12): TOTPAR was measured using a PR-NRS ranging from 0-10 (0=no relief, 10=complete relief). Total pain relief scores (TOTPARs) were calculated by multiplying the pain relief score at each postdose time point by the duration (in hours) since the preceding time point and then summing these values. The minimum value was 0, and the maximum value was 120. Higher scores was indicative of more pain relief.

| | Arm/Group Title | | | | |
|---|---|---|---|---|---|
| | | | | | Arm 5: |
| | | Arm 2: | Arm 3: Naproxen | Arm 4: Naproxen | Hydrocodone |
| | | Naproxen | Sodium 220 mg + | Sodium 440 mg + | 10 mg + |
| | Arm 1: | Sodium 440 | Acetaminophen | Acetaminophen | Acetaminophen |
| | Placebo | mg | 650 mg | 1000 mg | 650 mg |
| Least Squares | 18.20 | 69.96 | 76.80 (4.301) | 90.22 (4.190) | 57.03 (4.237) |
| Mean | (5.595) | (4.006) | | | |
| (Standard | | | | | |
| Error) | | | | | |

Time-Weighted Sum of Pain Intensity Difference Score From Baseline (0 Hour) to 8 Hours (SPID 0-8): Time-weighted sum of the pain intensity difference score was measured using a PI-NRS ranging from 0-10 (0=no pain, 10=very severe pain). The possible range of SPID for 0-8 hours was from −80 to 80. A higher value of SPID indicated greater pain relief. PID was the difference between baseline pain intensity and pain intensity at assessment (8 hours). Time-weighted sum of the pain intensity difference scores were derived by first multiplying each PID score by the time from the previous time point and adding these time-weighted PID scores together over the intervals from 0 to 8 hours.

| | Arm/Group Title | | | | |
|---|---|---|---|---|---|
| | | | | | Arm 5: |
| | | Arm 2: | Arm 3: Naproxen | Arm 4: Naproxen | Hydrocodone |
| | | Naproxen | Sodium 220 mg + | Sodium 440 mg + | 10 mg + |
| | Arm 1: | Sodium 440 | Acetaminophen | Acetaminophen | Acetaminophen |
| | Placebo | mg | 650 mg | 1000 mg | 650 mg |
| Least Squares | 3.24 | 34.11 | 39.98 (2.291) | 45.27 (2.232) | 29.58 (2.257) |
| Mean | (2.981) | (2.134) | | | |
| (Standard | | | | | |
| Error) | | | | | |

Participant's Global Evaluation of Study Medication OR Overall Impression of Study Medication According to Participant's Global Evaluation: Participants were asked to rate their overall impression of the study medication using the following scale: poor (0), fair (1), good (2), very good (3), and excellent (4) where higher score represented better outcome

| | Arm/Group Title | | | | |
|---|---|---|---|---|---|
| | | | | | Arm 5: |
| | | Arm 2: | Arm 3: Naproxen | Arm 4: Naproxen | Hydrocodone |
| | | Naproxen | Sodium 220 mg + | Sodium 440 mg + | 10 mg + |
| | Arm 1: | Sodium 440 | Acetaminophen | Acetaminophen | Acetaminophen |
| | Placebo | mg | 650 mg | 1000 mg | 650 mg |
| Least Squares | 0.8 (0.18) | 2.7 (0.13) | 2.9 (0.14) | 3.3 (0.13) | 2.7 (0.14) |
| Mean | | | | | |
| (Standard | | | | | |
| Error) | | | | | |

Pain Relief (PAR) Scores at Timepoints: Participants answered a question at individual time points: “how much relief do you have from your starting pain?” on a 11-point PR-NRS. Scale ranged from 0-no relief to 10-complete relief. Higher score indicated improvement in pain.

PAR Scores at Individual Timepoints. Least Squares Mean (Standard Error)

| | | | | | | Arm 5: |
|---|---|---|---|---|---|---|
| | | | Arm 2: | Arm 3: Naproxen | Arm 4: Naproxen | Hydrocodone |
| | | | Naproxen | Sodium 220 mg + | Sodium 440 mg + | 10 mg + |
| | | Arm 1: | Sodium 440 | Acetaminophen | Acetaminophen | Acetaminophen |
| | | Placebo | mg | 650 mg | 1000 mg | 650 mg |
| 0.25 | Hours | 0.45 (0.26) | 0.80 (0.18) | 1.36 (0.20) | 1.07 (0.19) | 1.04 (0.20) |
| 0.5 | Hours | 0.51 (0.44) | 2.27 (0.31) | 3.46 (0.33) | 4.03 (0.33) | 3.23 (0.36) |
| 0.75 | Hours | 0.77 (0.48) | 4.11 (0.35) | 5.10 (0.37) | 5.71 (0.36) | 5.08 (0.36) |
| 1 | Hours | 1.06 (0.49) | 5.29 (0.35) | 5.91 (0.38) | 6.60 (0.37) | 6.03 (0.37) |
| 1.25 | Hours | 1.13 (0.49) | 5.99 (0.35) | 6.46 (0.38) | 7.17 (0.37) | 6.40 (0.37) |
| 1.5 | Hours | 1.18 (0.48) | 6.36 (0.34) | 6.89 (0.37) | 7.65 (0.36) | 6.44 (0.36) |
| 2 | Hours | 1.08 (0.48) | 6.62 (0.34) | 7.37 (0.37) | 7.97 (0.36) | 6.38 (0.36) |
| 3 | Hours | 1.37 (0.49) | 6.52 (0.35) | 7.61 (0.38) | 8.24 (0.37) | 6.26 (0.37) |
| 4 | Hours | 1.66 (0.51) | 1.46 (0.36) | 7.68 (0.39) | 8.46 (0.38) | 5.96 (0.38) |
| 5 | Hours | 1.59 (0.52) | 6.45 (0.37) | 7.43 (0.40) | 8.23 (0.39) | 5.54 (0.40) |
| 6 | Hours | 1.53 (0.53) | 6.26 (0.38) | 7.23 (0.41) | 8.15 (0.40) | 4.88 (0.40) |
| 7 | Hours | 1.51 (0.55) | 6.05 (0.39) | 6.80 (0.42) | 7.94 (0.41) | 4.50 (0.41) |
| 8 | Hours | 1.65 (0.54) | 5.95 (0.39) | 6.63 (0.42) | 7.71 (0.41) | 4.23 (0.41) |
| 9 | Hours | 1.61 (0.56) | 5.73 (0.40) | 6.27 (0.43) | 7.44 (0.42) | 3.88 (0.43) |
| 10 | Hours | 1.73 (0.58) | 5.71 (0.42) | 5.66 (0.45) | 7.49 (0.44) | 3.66 (0.44) |
| 11 | Hours | 1.86 (0.60) | 5.71 (0.43) | 5.33 (0.46) | 7.26 (0.45) | 3.91 (0.46) |
| 12 | Hours | 1.89 (0.61) | 5.62 (0.44) | 5.18 (0.47) | 7.25 (0.46) | 3.98 (0.46) |

Pain Intensity Difference (PID) Scores at Individual Time Points: Pain Intensity was self-reported over 12 hours, using a pain rating of 0-10 on the PI-NRS, with score ranged from 0-10 (0=no pain; 10=worst imaginable pain). Pain intensity differences were calculated with respect to baseline at each time point after study drug administration.

PID Scores at Individual Timepoints. Least Squares Mean (Standard Error)

| | | | | | | Arm 5: |
|---|---|---|---|---|---|---|
| | | | Arm 2: | Arm 3: Naproxen | Arm 4: Naproxen | Hydrocodone |
| | | | Naproxen | Sodium 220 mg + | Sodium 440 mg + | 10 mg + |
| | | Arm 1: | Sodium 440 | Acetaminophen | Acetaminophen | Acetaminophen |
| | | Placebo | mg | 650 mg | 1000 mg | 650 mg |
| 0.25 | Hours | 0.25 (0.21) | 0.58 (0.15) | 1.01 (0.16) | 0.85 (0.15) | 00.79 (0.16) |
| 0.5 | Hours | 0.07 (0.33) | 1.82 (0.24) | 2.61 (0.25) | 3.06 (0.25) | 2.36 (0.25) |
| 0.75 | Hours | 0.24 (0.38) | 3.15 (0.27) | 3.82 (0.29) | 4.31 (0.29) | 3.77 (0.29) |
| 1 | Hours | 0.45 (0.39) | 3.97 (0.28) | 4.41 (0.30) | 4.95 (0.29) | 4.48 (0.30) |
| 1.25 | Hours | 0.34 (0.40) | 4.50 (0.28) | 4.87 (0.30) | 5.42 (0.30) | 4.71 (0.30) |
| 1.5 | Hours | 0.31 (0.39) | 4.72 (0.28) | 5.18 (0.30) | 5.76 (0.29) | 4.74 (0.30) |
| 2 | Hours | 0.22 (0.40) | 4.87 (0.28) | 5.49 (0.31) | 6.01 (0.30) | 4.67 (0.30) |
| 3 | Hours | 0.34 (0.41) | 4.75 (0.30) | 5.62 (0.32) | 6.17 (0.31) | 4.51 (0.31) |
| 4 | Hours | 0.53 (0.43) | 4.63 (0.31) | 5.64 (0.33) | 6.31 (0.32) | 4.26 (0.33) |
| 5 | Hours | 0.50 (0.44) | 4.59 (0.32) | 5.51 (0.34) | 6.13 (0.33) | 3.89 (0.34) |
| 6 | Hours | 0.47 (0.44) | 4.50 (0.32) | 5.28 (0.34) | 6.06 (0.33) | 3.39 (0.34) |
| 7 | Hours | 0.40 (0.45) | 4.36 (0.32) | 4.96 (0.34) | 5.82 (0.34) | 3.08 (0.34) |
| 9 | Hours | 0.53 (0.46) | 4.04 (0.33) | 4.46 (0.35) | 5.41 (0.34) | 2.56 (0.35) |
| 10 | Hours | 0.64 (0.40) | 4.01 (0.35) | 3.87 (0.37) | 5.43 (0.36) | 2.42 (0.37) |
| 11 | Hours | 0.69 (0.50) | 3.99 (0.35) | 3.63 (0.38) | 5.16 (0.37) | 2.71 (0.37) |
| 12 | Hours | 0.71 (0.50) | 3.91 (0.36) | 3.53 (0.38) | 5.16 (0.37) | 2.66 (0.38) |

No serious adverse events were reported.

1. A method of inducing minor pain relief in a human in need thereof comprising
administering to the human a pharmaceutical dosage form comprising acetaminophen in admixture with naproxen sodium, wherein the weight ratio of the acetaminophen to the naproxen sodium is from about 2:1 to about 4:1.

2. The method of claim 1, wherein the weight ratio of the acetaminophen to the naproxen sodium is about 3:1.

3. The method of claim 1, wherein the pharmaceutical dosage form comprises about 650 mg of acetaminophen and about 220 mg of naproxen sodium.

4. The method of claim 1, wherein the pain relief is induced within 15 minutes of the administration.

5. The method of claim 1, wherein the Pain Relief Score of the human at 15 minutes post-administration is greater, as compared to a Pain Relief Score of a human in need of minor pain relief at 15 minutes post-administration of a pharmaceutical dosage form comprising 1000 mg of acetaminophen and 440 mg of naproxen sodium.

6. (canceled)

7. (canceled)

8. (canceled)

9. (canceled)

10. (canceled)

11. (cancelled)

12. (canceled)

13. (cancelled)

14. (canceled)

15. (cancelled)

16. (canceled)

17. The method of claim 1, wherein the pharmaceutical dosage form is one or more tablets.

18. The method of claim 17, wherein the one or more tablets each comprise a core tablet.

19. The method of claim 18, wherein the core tablet comprises
about 10 wt. % to about 15 wt. % of naproxen sodium;
about 35 wt. % to about 45 wt. % of acetaminophen.
about 14 wt. % to about 18 wt. % of microcrystalline cellulose;
about 4 wt. % to about 8 wt. % lactose monohydrate; and
3 wt. % to about 7 wt. % crospovidone.

20. (canceled)

21. The method of claim 18, wherein the core tablet further comprises about 7 wt. % to about 12 wt. % of pregelatinized starch.

22. The method of claim 18, wherein the core tablet form further comprises about 7 wt. % to about 12 wt. % of silicified microcrystalline cellulose.

23. The method of claim 18, wherein the core tablet further comprises about 0.5 wt. % to about 1.5 wt. % of magnesium stearate.

24. (canceled)

25. (canceled)

26. The method of claim 17, wherein the one or more tablets are one or more coated tablets.

27. The method of claim 17, wherein the one or more tablets are one or more film-coated tablets.

28. The method of claim 1, wherein the pharmaceutical dosage form comprises two tablets, wherein each of the tablets comprises about 325 mg of acetaminophen and about 110 mg of naproxen sodium.

29. A method of making a core tablet comprising
granulating naproxen sodium, acetaminophen, microcrystalline cellulose, lactose monohydrate and crospovidone with a binder solution comprising purified water and pregelatinized starch, to form a granulation product;
combining the granulation product with silicified microcrystalline cellulose, crospovidone, and magnesium stearate to form a product blend; and
compressing the product blend to provide the core tablet.

30. The method of claim 29, wherein the core tablet comprises
about 10 wt. % to about 15 wt. % of naproxen sodium; and
about 35 wt. % to about 45 wt. % of acetaminophen.

31. (canceled)

32. (canceled)

33. (canceled)

34. (cancelled)

35. (canceled)

36. (cancelled)

37. The method of claim 29, wherein the core tablet comprises about 220 mg of naproxen sodium and 650 mg of acetaminophen.

38. The method of claim 29, further comprising coating the core tablet with a coating to form a coated tablet.

39. A core tablet produced according to the method of claim 29.

40. A core tablet comprising
an intragranular portion comprising acetaminophen and naproxen sodium, wherein the weight ratio of the acetaminophen to the naproxen sodium is from about 2:1 to about 4:1; and
an extragranular portion comprising a binder, a lubricant, a superdisintegrant, or a combination thereof.

41. A method of inducing minor pain relief in a human in need thereof comprising administering to the human the core tablet of claim 40.
